(19) United States
(12) Patent Application Publication
(10) Pub. No.: US 2006/0240495 A1
(43) Pub. Date: Oct. 26, 2006

(54) Non-invasive assessment of intra-amniotic environment

(75) Inventors: Irina A Buhimschi, Troy, MI (US); Robert Christner, Troy, MI (US); Catalin S. Buhimschi, Troy, MI (US); Carl P. Weiner, Troy, MI (US)

Correspondence Address: CIPHERGEN c/o FOLEY & LARDNER LLP, 3000 K STREET NW, SUITE 500, WASHINGTON, DC, 20007, US

(73) Assignee: Ciphergen Biosystems, Inc., Freemont, CA (US)

(21) Appl. No.: 10/541,793

(22) PCT Filed: Nov. 13, 2003
(86) PCT No.: PCT/US03/36118

§ 371 Date: Apr. 24, 2006

Related U.S. Application Data
(60) Provisional application No. 60/444,976, filed on Feb. 5, 2003.

(51) Int. Cl. G01N 33/53; G01N 33/00; G06F 19/00

(52) U.S. Cl. 435/7.92; 436/86; 702/19

(57) ABSTRACT

Assessing fetal health and maturity, and the integrity and health of the amnion, is effected via protein profiling of vaginal fluid, thereby to identify various abnormal conditions during pregnancy. This approach also provides a means for gauging the duration and/or magnitude of intraamniotic/fetal inflammation that occurs during pregnancy.

### BACKGROUND OF THE INVENTION

The present invention relates generally to the field of assessing fetal health and maturity and the integrity and health of the amnion. More particularly, it relates to a non-invasive method of identifying various abnormal conditions during pregnancy, and provides a means of assessing the duration and/or magnitude of intra-amniotic/fetal inflammation that occurs during pregnancy.

Rupture of the fetal membranes (ROM) precedes the onset of labor in approximately 10% of pregnant women at term. The natural history is that most of these women, approximately 60%, begin labor spontaneously within 24 hours of the rupture, and over 95% give birth within 72 hours. On the other hand, patients with premature rupture of the fetal membrane (PROM) usually present with a complaint of leaking fluid, vaginal discharge, vaginal bleeding, or pelvic pressure, but they are not in labor because uterine contractions, producing cervical effacement or dilation, are absent.

At term or close to term, and in the context of a fetus with pulmonary maturity, the central clinical question regarding management of women with PROM is whether to await spontaneous labor or to induce labor. The major risk both for the mother and the fetus is intrauterine infection, and the magnitude of the risk increases with the duration of ROM. The likelihood of a vaginal delivery is usually greatest when the onset of labor is spontaneous, but there is evidence that labor induction, as opposed to expectant management, decreases the risk of chorioamnionitis without increasing the overall cesarean delivery rate. If patients with clinically asymptomatic infection could be identified, it might be possible to lower the rate of cesarean section in these cases even further.

Preterm premature rupture of membranes (PPROM) refers to rupture of the fetal membranes before 37 weeks of gestation. PPROM is associated with some 30% of all preterm deliveries, 70% of spontaneous preterm labor prior to 28 weeks, and 10% of perinatal deaths. Despite extensive human and basic research, the etiology of most preterm births still remains unknown, and the frequency actually has increased over the past 2 decades, despite a myriad of nontargeted therapies. On a clinical level, preterm birth follows either preterm labor with intact membranes, or after labor preceded by PPROM.

Current therapeutic efforts to prevent preterm birth in women presenting with uterine contractions and intact membranes is confined to so-called tocolytic drugs that seek to inhibit the uterine contractions, but the efficacy of this approach has been disappointing in randomized trials, leading to a prolongation of pregnancy by only about 48 hours as compared to placebo. The management of preterm labor, when membranes are intact and there is no clinical chorioamnionitis, is far less challenging for the obstetrician than the management of preterm labor accompanied by PPROM and/or chorioamnionitis. While the goal in both instances is to prolong pregnancy to a point when the neonatal morbidity and mortality is minimal, the decision process for women with PPROM may take on a Faustian stance, since the longer the latency interval (time from rupture of membranes to labor), the greater the risk of fetal and maternal complications, particularly infection.

PPROM is often a manifestation of a preexisting microbial infection of the intrauterine amniotic cavity, which is found in 38% of all cases of PPROM at membrane rupture, and perhaps in as many as 70% should PPROM occur prior to 28 weeks gestation. The incidence of clinically obvious chorioamnionitis in PPROM ranges from 8-28% while the risk of maternal sepsis is only 2%. PPROM also increases the risk of maternal postpartum infections such as endometritis, septic thrombophlebitis and wound infections.

There are significant fetal consequences of PPROM, which include prematurity, infection, umbilical cord prolapse, skeletal deformation, pulmonary hypoplasia and an increase in overall perinatal mortality. Perinatal mortality approximates 44%, 11% and 5% when deliveries occur between 25-28 weeks, 29-32 weeks and 33-34 weeks, respectively. Mostly out of concern for maternal and fetal infection, PPROM was considered routinely an indication for expeditious delivery regardless of gestational age until just two decades ago. More recently, the management of PPROM prior to 32 weeks has evolved with the availability of more potent antibiotic agents and an improved understanding of the true risk of infection compared to the risk of prematurity, into an expectant approach, waiting for any sign of infection to initiate labor. The obstetrician's goal remains the optimization of the risk:benefit ratio, in which pregnancy is prolonged while the risk of infection is minimized.

A major frustration and limitation for the managing obstetrician of a patient with PPROM is the inability to assess the intrauterine environment directly and longitudinally for signs of infection or inflammation. In many clinical locales women with PPROM are not hospitalized, especially when the fetus is below the limit of viability (23-24 weeks). Rather, the women are instructed to monitor their temperature thrice daily and to report to the hospital if their temperature exceeds 38° C. The current diagnosis of chorioamnionitis is based solely on clinical signs and symptoms, which occur late in the natural evolution of the disease (e.g., maternal temperature greater than 38° C., fetal tachycardia, fundal tenderness, foul or purulent vaginal discharge, maternal tachycardia).

In some tertiary care centers, a transabdominal amniocentesis for amniotic fluid analysis is advocated to seek intra-amniotic infection before making a management decision. Two groups of tests are usually performed on the amniotic fluid sample. The first category includes the rapid tests, such as Gram stain, amniotic fluid glucose and white blood cell count, which are available at almost all hospitals and yield results in a short time. As a result, these tests have the most potential to influence management decisions in clinical settings. A positive Gram stain finding on an unspun fluid, a glucose concentration of less than 20 mg/dL and elevated WBC are all considered to be suggestive of intra-amniotic infection.

These tests have poor sensitivity and specificity, however, even when combined for the detection of microbial invasion of amniotic cavity. There is a high incidence of intrauterine infections with Mycoplasma and Ureaplasma that are not detected on Gram stain and do not lower the glucose concentration.

The second category of clinical tests consists of amniotic fluid cultures for aerobic or anaerobic organisms or Mycoplasma species. These tests may take a week or more for a final result. Because of the delay, they rarely change management. To circumvent the delay some have proposed one of several rapid tests on amniotic fluid often performed by an ELISA for the measurement of inflammatory mediators such as interleukin 6 (IL-6) and of neutrophil collagenase (MMP-8). None of these tests are used routinely in clinical practice, however, as they require special training and laborious experimental protocols, and even so are not completed in a timely enough fashion to impact on clinical management. It is not unusual that the results of these tests are contradictory, which adds to the confusion in electing the best clinical decision. Further, some 20% of amniocenteses performed for preterm PROM fail to yield a clinically useful sample. As a result of these limitations, most obstetricians do not perform amniocentesis reasoning the results are unlikely to have any impact on management.

It is a critical action on every labor suite every day to confirm or refute the diagnosis of membrane rupture. Yet, vaginal assessment is the product of tests that are expensive, lengthy, and poorly reproducible. When present, the visualization of clear fluid coming from the cervical Os (pooling) is the most reliable sign of membrane rupture. When pooling is not obvious, or the pregnancy is complicated by oligo or anhydramnios, a drop of the vaginal fluid can be smeared on a glass slide, allowed to dry on a glass slide, and viewed with a light microscope (fern test). Amniotic fluid crystallizes to form a “fern-like” pattern due to the high relative concentrations of sodium chloride, proteins, and carbohydrates. Alternatively, or additionally, a drop of amniotic fluid can be placed on a dry piece of nitrazine paper (nitrazine test). The nitrazine test is a pH measurement that is based on the fact that amniotic fluid has a pH between 7-7.5, much higher than normal acidic vaginal fluid. The presence of amniotic fluid turns the nitrazine paper blue. A careful history, together with the results of the nitrazine and fern tests are said to have a 91% sensitivity and 96% specificity in diagnosing rupture of the membranes, but there is no gold standard. Further, false negative tests are common with preterm PROM.

While both the fern test and the nitrazine test are rapid and inexpensive, false positive results occur as a result of contamination with heavy vaginal discharge, blood, cervical mucus, semen, alkaline urine or soap. False negative results occur when small volumes of fluid leak and are more common with prolonged rupture of membranes (longer than 24 hours). When the diagnosis remains in question, the amniotic fluid index (AFI) is sometimes considered, but there are many other causes of low amniotic fluid, and an intermittent leak is often associated with normal fluid volumes.

When doubt still exists, 5 mL of indigo carmine dye diluted in sterile saline can be instilled into the amniotic cavity transabdominally, and staining on a vaginal tampon or a sanitary pad indicates leakage of fluid (amnio dye test). This definitive test is invasive and may also cause maternal discomfort, inadvertent puncture of the umbilical cord and rupture of previously intact membranes. False positive results may occur secondary to contamination of the sanitary pad with urine as indigo carmine is excreted by the kidney and causes a blue discoloration of the urine as well.

The value of identifying in the vaginal fluid of women with suspected PROM high concentrations of certain proteins present in high, amounts in amniotic fluid forms the basis for tests that assess the existence of PROM. Immunoreactive fetal fibronectin, insulin-like growth factor binding protein-1, alpha-fetoprotein, prolactin, and human placental lactogen, as well as diamino-oxidase enzymatic activity, have all been studied as diagnostic tools in vaginal fluid with mixed results.

In addition to an ability to predict PROM, it would be extremely useful to be able to assess inflammation in the intra-amniotic environment. For many years physicians have assumed that the morbidity and mortality associated with preterm delivery was a direct result of the early gestational age. It is now known, however, that many preterm deliveries result because of fetal illness, and it is the illness that is the proximate cause of the morbidity such as cerebral palsy. Many of these illnesses share inflammation as a common pathway, and the present inventors have discovered that it is the inflammation that triggers labor.

All available tocolytic agents fail to prolong gestation more than a few days compared to placebo. In some respects, this could be a saving grace since it may be worse for a fetus to have its in utero time prolonged if it is ill.

It is not known how long, if at all, a fetus can tolerate an inflammatory response. Presumably a fetus will have a better chance of survival if it is delivered before any damage from inflammation has occurred. This would require a means of measuring inflammation at a very early stage, before clinical signs of inflammation are present. In cases where inflammation occurs before the fetus can survive outside the womb, early detection of inflammation allows treatment of the fetus in utero, such as by administering free radical traps to reduce oxygen free radical toxicity. Some of these substances cross the placenta and thus fetal treatment can be accomplished by giving the drug to the pregnant woman. In cases where the decision is made to deliver the fetus, treatment of the infant for inflammation can begin immediately, since there is no reason to believe the inflamed preterm newborn is no longer at risk. Indeed, several lines of study indicate a stimulus can trigger an ongoing inflammatory process even when the stimulus is removed. At present, there is no easy test for identifying evidence of inflammation in newborns, in spite of the fact that early identification would permit the timely initiation of postnatal therapy.

On the other hand, if the inflammation has been ongoing for some time or is chronic, then prematurity may be the greater risk, particularly if the fetus already is damaged. There is at present, however, no test to determine fetal inflammation, and more particularly there is no test that is capable of distinguishing between short term or recent inflammation and long term or chronic inflammation. Without a means for identifying which fetuses are inflamed, and which fetuses are not, and the duration or magnitude of the inflammation, early intervention is not a possibility.

Obstetricians assume considerable legal liability for the outcome of a pregnancy. In the absence of knowledge, they have routinely been blamed for adverse outcomes. Thus, a means of identifying high-risk situations existing prior to delivery by the obstetrician is highly desirable.

It is highly desirable to provide a test that is capable of assessing PROM and other intra-amniotic abnormalities, particularly a test capable of assessing the duration and/or magnitude of intra-amniotic/fetal inflammation. The test should be both highly accurate and capable of being performed simply and rapidly in the hospital, without requiring that a sample be sent to a remote laboratory. The fern test and nitrazine test are rapid and inexpensive indicators of PROM, but not of other intra-amniotic abnormalities, and they are not highly accurate. Rapid tests using strips impregnated with immobilized antibodies against alpha-fetoprotein or insulin-like growth factor binding protein-1 have been developed. Compared to the nitrazine test and fern test, they are more expensive, do not improve accuracy of the diagnosis and are not used clinically.

### SUMMARY OF THE INVENTION

To address the above-described shortcomings of conventional approaches in this area, the present invention provides a non-invasive method for assessing the intra-amniotic environment, which method is simple and rapid to perform and is accurate. In one embodiment of the invention, the method entails obtaining a vaginal sample from a pregnant subject, typically by swabbing the vagina with a cotton swab. The swab may be inserted into a liquid, typically a buffer a solution, to provide a sample for analysis. The sample is analyzed, to determine the presence or absence in the sample of a plurality of biomarkers that are indicative of status of the intra-amniotic environment. Results from the assessment of the vaginal sample informs a diagnostic or prognostic determination in relation to the subject. In another embodiment, the steps of obtaining and analyzing the sample are repeated at least a second time.

The inventive method may comprise an ELISA, but more preferably it comprises mass spectrometric analysis effected via Surface Enhanced Laser/Desorption ionization (SELDI). The latter technique is particularly preferred when a plurality of biomarkers is being assessed. When SELDI is used, the method comprises applying the vaginal sample to a biochip comprising at least one absorbent, such as a hydrophobic adsorbent or a cation exchange absorbent. The biochip is subjected to mass spectrometric analysis, and mass-spectrometry peak data are obtained for the vaginal sample. These data are processed by means of software that includes an algorithm for analyzing information extracted from a spectrum. Thus, the software algorithm implements a pattern-recognition analysis that is keyed to data relating to at least one of the biomarkers, according to the present invention.

Biomarkers indicative of various conditions may be determined. For example, the presence or absence of biomarkers indicative of rupture of the fetal membrane, intra-amniotic infection, intra-amniotic inflammation, and fetal lung maturation may be determined. In one embodiment the biomarkers are selected from the group consisting of alpha-fetoprotein, fetal fibronectin, insulin-like growth factor binding protein-1, prolactin and human placental lactogen, and fragments thereof. Other biomarkers include beta-2-microglobulin and cystatin-C, and fragments thereof. Still other biomarkers are calgranulins or defensins, or fragments thereof.

It is preferable to collect and analyze a first vaginal sample early during a pregnancy, to provide a baseline against which subsequent vaginal samples are compared. Abnormal clinical status includes PROM, intra-amniotic infection, and intra-amniotic inflammation. If a subsequent sample indicates that an abnormal clinical status is present, then the assessment can include a recommendation for treatment. The treatment can be monitored by assaying at least one vaginal sample during treatment, to determine the presence or absence in the vaginal sample of biomarkers that are indicative of status of the intra-amniotic environment. Based on the results, antibiotic treatment, tocolytic treatment, anti-inflammatory treatment, or antioxidant treatment may be recommended. Alternatively, induction of labor or a cesarean section may be recommended based on the results. In this case, a vaginal sample may be analyzed for biomarkers that are indicative of fetal health and maturity, particularly fetal lung maturation.

Early intervention is not possible without a means for identifying which fetuses are inflamed and which are not, and for gauging the duration or magnitude of the inflammation. Accordingly, the present invention also provides a test determining fetal inflammation, particularly to distinguish between short term or recent inflammation and long term or chronic inflammation.

Thus, another embodiment of the present invention entails evaluating the duration and/or magnitude of intra-amniotic or fetal inflammation. This can be determined by obtaining a vaginal sample from an individual and subjecting the sample to analysis to determine the presence or absence in the sample of one or more oxidized or carbonylated proteins or peptides, which are indicative of inflammation. The results from the assessment of the vaginal sample again inform a diagnostic or prognostic determination in relation to the subject. To carry out this aspect of the invention, a vaginal sample may be treated with dinitrophenol which is incorporated into the oxidized or carbonylated protein or peptide. Either an ELISA or mass spectrometric analysis effected via SELDI may be used to detect the presence of the oxidized or carbonylated proteins or peptides. For example, total carbonyl content of the oxidized or carbonylated peptides can be measured, pursuant to the invention, by derivatizing the peptides with dintrophenylhydrazine.

As in other embodiments described above, an applicable SELDI analysis in this context comprises the use of a biochip, and analysis comprises subjecting mass-spectrometry peak data obtained for the vaginal sample to software analysis comprised of an algorithm for analyzing data extracted from a spectrum, which implements a pattern-recognition analysis that is keyed to data relating to one or more oxidized or carbonylated proteins or peptides. Preferably, a first vaginal sample is collected early during a pregnancy and contributes to a baseline against which subsequent vaginal samples are compared.

The determination of the presence of inflammation affords important input to the clinician for formulating treatment recommendations. Treatments in addition to those just mentioned may be appropriate in these situations. Detection of inflammation at a very early stage, before clinical signs of inflammation are present, increases the likelihood that an appropriate treatment will be implemented. In cases where inflammation occurs before the fetus can survive outside the womb, early detection of inflammation allows treatment of the fetus in utero, such as by administering free radical traps to reduce oxygen free radical toxicity. Since some of these substances cross the placenta, fetal treatment can be accomplished by giving the drug to the pregnant woman. In cases where the decision is made to deliver the fetus, treatment of the infant for inflammation can begin immediately. On the other hand, if the inflammation has been ongoing for some time or is chronic, then prematurity may be the greater risk, particularly if the fetus already is damaged, and the recommendation then would likely be to maintain the pregnancy.

When SELDI analysis is used to detect the presence or absence of oxidized or carbonylated proteins or peptides, the vaginal sample is treated with dinitrophenol, and then is applied to a biochip comprising an anti-dinitrophenol antibody and subjected to mass spectrometric analysis that is keyed to a shift in molecular weight, relative to a sample not treated with dinitrophenol, that corresponds to the incorporated dinitrophenol group. Alternatively, the dinitrophenol treated vaginal sample is applied to a biochip comprising, an anti-dinitrophenol antibody and subjected to mass spectrometric analysis is keyed to a shift or approximately 16 Da, relative to a sample not treated with dinitrophenol, that corresponds to the molecular mass of oxygen.

In accordance with another embodiment, the present invention provides methodology for qualifying status of the intra-amniotic environment in a subject over time. This approach involves (i) providing spectra generated by mass spectrometric analysis of at least two vaginal samples taken from the subject and (ii) extracting data from the spectra and subjecting the data to pattern-recognition analysis that is keyed to at least two peaks in the spectra.

Yet another embodiment of the invention relates to a kit for detecting, from a sample of vaginal fluid, the presence of at least two biomarkers indicative of status of the intra-amniotic environment, comprising (a) a substrate adapted for inserting into a mass spectrophotometer for analysis, and (b) instructions for applying a sample of vaginal fluid to the substrate and subjecting the substrate to mass spectrometric analysis. The substrate may be a biochip, such as a biochip having a hydrophobic adsorbent, a cation exchange adsorbent, or an anti-dinitrophenol absorbent. The kit additionally may include, in a separate container, a quantity of the biomarker in pure form to be used as a standard. The kit also may include a washing solution for removing unbound material from the substrate.

A further embodiment of the invention is a kit for detecting, from a sample of vaginal fluid, the presence of at least one oxidized or carbonylated peptide indicative of status of the intra-amniotic environment. This kit includes (a) a substrate that binds the peptide, and (b) instructions for applying a sample of vaginal fluid to the substrate and subjecting the substrate to analysis. The substrate may be an ELISA substrate, or it may be a substrate adapted for insertion into a mass spectrophotometer for analysis. The kit additionally can include, in separate container, a quantity of the oxidized or carbonylated peptide in pure form to be used as a standard. The kit also may include a washing solution for removing unbound material from the substrate.

In still another embodiment, the present invention provides for identifying biomarkers that are present in vaginal fluid and that are indicative of status of the intra-amniotic environment. The method in this regard comprises (a) profiling a sample of vaginal fluid by mass spectrophotometric analysis, (b) profiling a sample of amniotic fluid by mass spectrophotometric analysis, and (c) comparing the profiles obtained in (a) and (b) to identify biomarkers in vaginal fluid that also are found in amniotic fluid. The method may include as well a correlating, to a clinical status, of the presence or absence of biomarkers in the vaginal fluid that also are found in the amniotic fluid. Illustrative of a correlated clinical status in this regard is rupture of the fetal membrane, intra-amniotic infection, and intra-amniotic inflammation.

Also an embodiment of the invention is an alternative methodology for identifying biomarkers that are present in vaginal fluid and that indicate status of the intra-amniotic environment. This approach comprises (a) profiling a first sample of vaginal fluid from a subject having a normal pregnancy by mass spectrophotometric analysis, (b) profiling a second sample of vaginal fluid from a subject having a pregnancy characterized by an abnormal clinical status by mass spectrophotometric analysis, and (c) correlating the presence or absence of the biomarkers in the vaginal fluid to clinical status of the pregnancy.

### BRIEF DESCRIPTION OF THE DRAWINGS

FIG. 1 is a diagrammatic representation of the model for identifying a biomarker profile for diagnosing intra-amniotic inflammation (IAI) from a sample of vaginal fluid.

FIG. 2 is a bar graph comparing concentrations of beta-2-microglobulin and cystatin C in samples of amniotic fluid and vaginal fluid in patients with PROM.

FIG. 3 is a series of profiles of amniotic and vaginal fluid from two women with intra-amniotic inflammation.

FIG. 4 are vaginal protein profiles in a patient with PROM, taken on the day of rupture (A) and after three days (B).

### DETAILED DESCRIPTION OF THE PREFERRED EMBODIMENTS

Definitions

“Gas phase ion spectrometer” refers to an apparatus that detects gas phase ions. Gas phase ion spectrometers include an ion source that supplies gas phase ions. Gas phase ion spectrometers include, for example, mass spectrometers, ion mobility spectrometers, and total ion current measuring devices. “Gas phase ion spectrometry” refers to the use of a gas phase ion spectrometer to detect gas phase ions.

“Mass spectrometer” refers to a gas phase ion spectrometer that measures a parameter which can be translated into mass-to-charge ratios of gas phase ions. Mass spectrometers generally include an ion source and a mass analyzer. Examples of mass spectrometers are time-of-flight, magnetic sector, quadrupole, filter, ion trap, ion cyclotron resonance, electrostatic sector analyzer and hybrids of these. “Mass spectrometry” refers to the use of a mass spectrometer to detect gas phase ions.

“Laser desorption mass spectrometer” refers to a mass spectrometer which uses laser as a means to desorb, volatilize, and ionize an analyte.

“Tandem mass spectrometer” refers to any mass spectrometer that is capable of performing two successive stages of m/z-based discrimination or measurement of ions, including of ions in an ion mixture. The phrase includes mass spectrometers having two mass analyzers that are capable of performing two successive stages of m/z-based discrimination or measurement of ions tandem-in-space. The phrase further includes mass spectrometers having a single mass analyzer that are capable of performing two successive stages of m/z-based discrimination or measurement of ions tandem-in-time. The phrase thus explicitly includes Qq-TOF mass spectrometers, ion trap mass spectrometers, ion trap-TOF mass spectrometers, TOF-TOF mass spectrometers, and Fourier transform ion cyclotron resonance mass spectrometers, electrostatic sector—magnetic sector mass spectrometers, and combinations thereof.

“Mass analyzer” refers to a sub-assembly of a mass spectrometer that comprises means for measuring a parameter which can be translated into mass-to-charge ratios of gas phase ions. In a time-of flight mass spectrometer the mass analyzer comprises an ion optic assembly, a flight tube and an ion detector.

“Ion source” refers to a sub-assembly of a gas phase ion spectrometer that provides gas phase ions. In one embodiment, the ion source provides ions through a desorption/ionization process. Such embodiments generally comprise a probe interface that positionally engages a probe in an interrogatable relationship to a source of ionizing energy (e.g., a laser desorption/ionization source) and in concurrent communication at atmospheric or subatmospheric pressure with a detector of a gas phase ion spectrometer.

Forms of ionizing energy for desorbing/ionizing an analyte from a solid phase include, for example: (1) laser energy; (2) fast atoms (used in fast atom bombardment); (3) high energy particles generated via beta decay of radionuclides (used in plasma desorption); and (4) primary ions generating secondary ions (used in secondary ion mass spectrometry). The preferred form of ionizing energy for solid phase analytes is a laser (used in laser desorption/ionization), in particular, nitrogen lasers, Nd-Yag lasers and other pulsed laser sources. “Fluence” refers to the laser energy delivered per unit area of interrogated image. Typically, a sample is placed on the surface of a probe, the probe is engaged with the probe interface and the probe surface is struck with the ionizing energy. The energy desorbs analyte molecules from the surface into the gas phase and ionizes them.

Other forms of ionizing energy for analytes include, for example: (1) electrons which ionize gas phase neutrals; (2) strong electric field to induce ionization from gas phase, solid phase, or liquid phase neutrals; and (3) a source that applies a combination of ionization particles or electric fields with neutral chemicals to induce chemical ionization of solid phase, gas phase, and liquid phase neutrals.

“Probe” in the context of this invention refers to a device adapted to engage a probe interface and to present an analyte to ionizing energy for ionization and introduction into a gas phase ion spectrometer, such as a mass spectrometer. A “probe” will generally comprise a solid substrate (either flexible or rigid) comprising a sample presenting surface on which an analyte is presented to the source of ionizing energy.

“Surface-enhanced laser desorption/ionization” or “SELDI” is a method of gas phase ion spectrometry (e.g., mass spectrometry) in which the surface of the probe that presents the analyte to the energy source plays an active role in desorption/ionization of analyte molecules. SELDI technology is described, e.g., in U.S. Pat. No. 5,719,060 (Hutchens and Yip) and U.S. Pat. No. 6,225,047 (Hutchens and Yip).

One version of SELDI, called “Surface-Enhanced Affinity Capture” or “SEAC” involves the use of probes comprising a chemically selective surface. (“SELDI probe.”) “Chemically selective surface” refers to a surface to which is bound either an adsorbent (also called a “capture reagent”) or a reactive moiety that is capable of binding a capture reagent, e.g., through a reaction forming a covalent or coordinate covalent bond.

“Reactive moiety” refers to a chemical moiety that is capable of binding a capture reagent. Epoxide and carbodiimidizole are useful reactive moieties to covalently bind polypeptide capture reagents such as antibodies or cellular receptors. Nitriloacetic acid and iminodiacetic acid are useful reactive moieties that function as chelating agents to bind metal ions that interact non-covalently with histidine containing peptides. “Reactive surface” refers to a surface to which a reactive moiety is bound.

“Adsorbent” or “capture reagent” refers to any material capable of binding an analyte (e.g., a target polypeptide). “Chromatographic adsorbent” refers to a material typically used in chromatography. Chromatographic adsorbents include, for example, ion exchange materials, metal chelators, immobilized metal chelates, hydrophobic interaction adsorbents, hydrophilic interaction adsorbents, dyes, mixed mode adsorbents (e.g., hydrophobic attraction/electrostatic repulsion adsorbents). “Biospecific adsorbent” refers an adsorbent comprising a biomolecule, e.g., a nucleotide, a nucleic acid molecule, an amino acid, a polypeptide, a simple sugar, a polysaccharide, a fatty acid, a lipid, a steroid or a conjugate of these (e.g., a glycoprotein, a lipoprotein, a glycolipid). In certain instances the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than a chromatographic adsorbent. Further examples of adsorbents for use in SELDI can be found in U.S. Pat. No. 6,225,047 (Hutchens and Yip, “Use of retentate chromatography to generate difference maps,” May 1, 2001). “Adsorbent surface” refers to a surface to which an adsorbent is bound.

Another version of SELDI, called “Surface-Enhanced Neat Desorption” or “SEND” involves the use of probes comprising energy absorbing molecules chemically bound to the probe surface. (“SEND probe.”) “Energy absorbing molecules” (“EAM”) refer to molecules that are capable of absorbing energy from a laser desorption ionization source and thereafter contributing to desorption and ionization of analyte molecules in contact therewith. The phrase includes molecules used in MALDI, frequently referred to as “matrix”, and explicitly includes cinnamic acid derivatives, sinapinic acid (“SPA”), cyano-hydroxy-cinnamic acid (“CHCA”) and dihydroxybenzoic acid, ferulic acid, hydroxyacetophenone derivatives, as well as others. It also includes EAMs used in SELDI. SEND is further described in U.S. Pat. No. 5,719,060 and U.S. patent application 60/408,255, filed Sep. 4, 2002 (Kitagawa, “Monomers And Polymers Having Energy Absorbing Moieties Of Use In Desorption/Ionization Of Analytes”).

Another version of SELDI, called “Surface-Enhanced Photolabile Attachment and Release” or “SEPAR” involves the use of probes having moieties attached to the surface that can covalently bind an analyte, and then release the analyte through breaking a photolabile bond in the moiety after exposure to light, e.g., laser light.

“Adsorption” refers to detectable non-covalent binding of an analyte to an adsorbent or capture reagent.

“Eluant” or “wash solution” refers to an agent, typically a solution, which is used to affect or modify adsorption of an analyte to an adsorbent surface and/or remove unbound materials from the surface. The elution characteristics of an eluant can depend, for example, on pH, ionic strength, hydrophobicity, degree of chaotropism, detergent strength and temperature.

“Analyte” refers to any component of a sample that is desired to be detected. The term can refer to a single component or a plurality of components in the sample.

The “complexity” of a sample adsorbed to an adsorption surface of an affinity capture probe means the number of different protein species that are adsorbed.

“Molecular binding partners” and “specific binding partners” refer to pairs of molecules, typically pairs of biomolecules that exhibit specific binding. Molecular binding partners include, without limitation, receptor and ligand, antibody and antigen, biotin and avidin, and biotin and streptavidin.

“Monitoring” refers to recording changes in a continuously varying parameter.

“Biochip” refers to a solid substrate having a generally planar surface to which a capture reagent (adsorbent) is attached. Frequently, the surface of the biochip comprises a plurality of addressable locations, each of which location has the capture reagent bound there. Biochips can be adapted to engage a probe interface and, therefore, function as probes.

“Protein biochip” refers to a biochip adapted for the capture of polypeptides. Many protein biochips are described in the art. These include, for example, protein biochips produced by Ciphergen Biosystems (Fremont, Calif.), Packard BioScience Company (Meriden Conn.), Zyomyx (Hayward, Calif.) and Phylos (Lexington, Mass.). Examples of such protein biochips are described in the following patents or patent applications: U.S. Pat. No. 6,225,047 (Hutchens and Yip, “Use of retentate chromatography to generate difference maps,” May 1, 2001); International publication WO 99/51773 (Kuimelis and Wagner, “Addressable protein arrays,” Oct. 14, 1999); U.S. Pat. No. 6,329,209 (Wagner et al., “Arrays of protein-capture agents and methods of use thereof,” Dec. 11, 2001) and International publication WO-00/56934 (Englert et al., “Continuous porous matrix arrays,” Sep. 28, 2000).

Protein biochips produced by Ciphergen Biosystems comprise surfaces having chromatographic or biospecific adsorbents attached thereto at addressable locations. Ciphergen ProteinChip® arrays include NP20, H4, H50, SAX-2, WCX-2, IMAC-3, LSAX-30, LWCX-30, IMAC-40, PS-10, PS-20 and PG-20. These protein biochips comprise an aluminum substrate in the form of a strip. The surface of the strip is coated with silicon dioxide.

In the case of the NP-20 biochip, silicon oxide functions as a hydrophilic adsorbent to capture hydrophilic proteins.

H4, H50, SAX-2, WCX-2, IMAC-3, PS-10 and PS-20 biochips further comprise a functionalized, cross-linked polymer in the form of a hydrogel physically attached to the surface of the biochip or covalently attached through a silane to the surface of the biochip. The H4 biochip has isopropyl functionalities for hydrophobic binding. The H50 biochip has nonylphenoxy-poly(ethylene glycol)methacrylate for hydrophobic binding. The SAX-2 biochip has quaternary ammonium functionalities for anion exchange. The WCX-2 biochip has carboxylate functionalities for cation exchange. The IMAC-3 biochip has nitriloacetic acid functionalities that adsorb transition metal ions, such as Cu++ and Ni++, by chelation. These immobilized metal ions allow adsorption of peptide and proteins by coordinate bonding. The PS-10 biochip has carboimidizole functional groups that can react with groups on proteins for covalent binding. The PS-20 biochip has epoxide functional groups for covalent binding with proteins. The PS-series biochips are useful for binding biospecific adsorbents, such as antibodies, receptors, lectins, heparin, Protein A, biotin/streptavidin and the like, to chip surfaces where they function to specifically capture analytes from a sample. The PG-20 biochip is a PS-20 chip to which Protein G is attached. The LSAX-30 (anion exchange); LWCX-30 (cation exchange) and IMAC-40 (metal chelate) biochips have functionalized latex beads on their surfaces. Such biochips are further described in: WO 00/66265 (Rich et al., “Probes for a Gas Phase Ion Spectrometer,” Nov. 9, 2000); WO 00/67293 (Beecher et al., “Sample Holder with Hydrophobic Coating for Gas Phase Mass Spectrometer,” Nov. 9, 2000); U.S. patent application 09/908,518 (Pohl et al., “Latex Based Adsorbent Chip,” Jul. 16, 2002) and U.S. patent application 60/350,110 (Um et al., “Hydrophobic Surface Chip,” Nov. 8, 2001).

Upon capture on a biochip, analytes can be detected by a variety of detection methods including for example, gas phase ion spectrometry methods, optical methods, electrochemical methods, atomic force microscopy and radio frequency methods. Gas phase ion spectrometry methods are described herein. Of particular interest is the use of mass spectrometry and, in particular, SELDI. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltammetry and amperometry methods. Radio frequency methods include multipolar resonance spectroscopy.

### DESCRIPTION OF THE INVENTION

In accordance with the present invention, the vaginal environment is monitored in order to assess the status of the fetus and of intra-amniotic environment. This provides a non-invasive means of assessing both fetal health and maturity and the integrity and health of the amnion. In particular, the present invention provides a non-invasive means of identifying premature rupture of fetal membranes (PROM). Left untreated, PROM and other conditions can lead to premature delivery.

More particularly, the present invention relates to the presence or absence of specific biomarkers or combinations of biomarkers that are indicative of fetal health and maturity and/or the status of the amniotic environment. In the present context, a biomarker is an organic biomolecule, particularly a polypeptide or protein, which is differentially present in a sample taken from a subject having a particular abnormal clinical status in the amniotic environment as compared to a comparable sample taken from a “normal” subject that does not have the abnormal clinical status. An abnormal clinical status includes such conditions as intra-amniotic inflammation or infection, and PROM. It may also be possible to detect biomarkers indicative of amniotic fluid embolism in samples of maternal blood. A biomarker also may be a biomolecule that is indicative of fetal health or maturity.

The present invention detects biomarkers that are differentially present in samples taken from normal patients versus those with an abnormal clinical status. A biomarker is “differentially present,” in samples taken from normal patients and those with an abnormal clinical status, if the biomarker is present at an elevated level or a decreased level in samples of the latter patients as compared to samples of normal patients. In a preferred embodiment, a biomarker is a polypeptide that is characterized by an apparent molecular weight, as determined by gas phase ion spectrometry, and that is present in samples from subjects with abnormal clinical status in an elevated or decreased level, as compared to normal subjects.

Novel biomarkers that are present in vaginal fluid and that are indicative of the status of fetal health and/or health or integrity of the amnion can be identified by profiling a sample of vaginal fluid by mass spectrophotometric analysis, profiling a contemporaneously-obtained sample of amniotic fluid by mass spectrophotometric analysis, and comparing the profiles obtained in order to identify biomarkers in vaginal fluid that also are found in amniotic fluid. In a preferred embodiment, the method additionally entails correlating the presence or absence of the biomarkers in the vaginal fluid that are also found in the amniotic fluid to a clinical status. Alternatively, novel biomarkers can be identified by profiling a first sample of vaginal fluid from a subject having a normal pregnancy by mass spectrophotometric analysis, profiling a second sample of vaginal fluid from a subject having a pregnancy characterized by an abnormal clinical status by mass spectrophotometric analysis, and correlating the presence or absence of the biomarkers in the vaginal fluid to clinical status of the pregnancy. This latter approach can identify biomarkers that are not found in amniotic fluid, such as biomarkers that are degradation products of amniotic proteins, or biomarkers that are produced by the vagina in response to the presence of amniotic fluid.

The biomarkers of the invention are capable of identifying PROM, intra-amniotic inflammation and/or infection, fetal lung maturation, amniotic fluid embolism. A single biomarker or combination of biomarkers can be used, but a plurality of biomarkers is preferred. The biomarkers and combinations of biomarkers thus can be used to qualify the risk of preterm delivery in a patient.

Examples of biomarkers that are indicative of intra-amniotic inflammation as the abnormal clinical status include the defensins and calgranulins disclosed in copending application Ser. No. 60/426,096, the contents of which are incorporated herein in their entirety by reference. Calgranulins are members of the S100 group of proteins, which are calcium-binding proteins that contain two canonical EF-hand structural motifs. They have received increasing attention due to their possible involvement in diseases such as Alzheimer's, cancer, cardiomyopathy, psoriasis, rheumatoid arthritis, and other inflammatory disorders. S100 A8 (calgranulin A) and S100 A9 (calgranulin B) can combine to form homodimers and heterodimers, which also have antimicrobial properties. The three principle human neutrophil defensins, HNP 1-3, belong to the family of unique to neutrophils and account for 99 percent of the defensin content in these cells. HNP-1, -2 and -3 belong to the family of cationic, trifsulfide-containing microbicidal peptides. Their production and release is induced by cytokines and microbial products such as lipopolysaccharide, a component of the cell wall of Gram negative bacteria. In preferred embodiments, the calgranulin is calgranulin A or calgranulin C and the defensin is HNP-1 (alpha-defensin 1) or HNP-2 (alpha-defensin 2). Other exemplary biomarkers are beta-2-microglobulin and cystatin-C.

In a preferred embodiment, the biomarkers are oxidized or carbonylated peptides. It has been discovered that in preterm labor associated with inflammation there is an imbalance between the production and defense against free radicals, and that these free radicals produce oxidized or carbonylated proteins. Thus, the presence of oxidized or carbonylated proteins in amniotic fluid is an indicator of chronicity.

While all biomarkers in accordance with the present invention are reliable predictors of one or more aspects of fetal and health and integrity of the amnion, oxidized and carbonylated proteins additionally provide information about the intensity and/or duration of the insult. The reason for this is that in order for protein oxidation to occur, the insult must have been present for a sufficient time or have been of a sufficient intensity to deplete the antioxidant reserves. This has been confirmed by an experiment in which amniotic fluid was exposed to peroxyl free radicals generated in vitro by the spontaneous decomposition of 2,2′ azobis-2-methylpropionamidine dihydrochloride (ABPA). Protein carbonylation and protein fragmentation were assessed as indicators of free radical damage, and total carbonyl content was measured by derivatization with dinitrophenylhydrazine (DNPH) followed by Western blotting for DNPH-derivatized proteins using anti-dinitrophenyl DNP antibodies before and after exposure to ABPA. A dose and time-dependent increase in protein carbonylation in amniotic fluid in response to ABPA was revealed. Protein carbonylation was associated with substantial protein degradation which was substantially higher in amniotic fluid than in fetal plasma.

Protein oxidation or carbonylation in amniotic fluid thus is a predictor not only of the existence of intra-amniotic inflammation, but also of the intensity and duration of this condition. Incorporation of dinitrophenol (DNP) groups into the oxidized and carbonylated proteins that are found in vaginal fluid as a result of intra-amniotic inflammation provides a means of measuring total carbonyl content, and hence intensity and/or duration of the insult. The median amount of DNP is significantly greater in patients with intra-amniotic inflammation than in the groups without intra-amniotic inflammation. Thus, the preterm fetus of mothers with intra-amniotic inflammation is exposed to a highly oxidative environment. Furthermore, when intra-amniotic inflammation was estimated by MR score, as defined herein, protein oxidation was significantly higher in samples of amniotic fluid that had MR scores >2. Lack of a minimal level of protein carbonylation is indicative either of lack of inflammation, if it co-exists with a low MR score, or of an inflammation that has been present for a long time, if it coexists with a high MR score.

These data show that measurement of protein oxidation or carbonylation in vaginal fluid is a valuable source of information about the extent and gravity of the inflammatory insult that may correlate more reliably with fetal outcome than the presence of inflammation per se. While the DNP incorporation and immunoassay method just described can be used to measure total carbonyl content in these proteins, the presence and extent of protein oxidation or carbonylation also can be followed using SELDI analysis as described herein. In this context, a biochip coated with a DNP capture antibody is used.

The present invention provides a rapid and reliable proteomic approach to identifying conditions which can lead to preterm delivery, such as intra-amniotic inflammation and/or infection. This is the first proteomic characterization based on the existence of multiple predictors of amniotic status in vaginal fluid. Detailed analyses of the biomarkers permits characterization and quantitative validation of the changes involved.

The present invention also provides a rapid and reliable proteomic approach to determining whether a fetus has sufficient lung function to ensure survival outside the womb. During the second trimester of pregnancy fetal lung liquid constitutes a major inflow into the amniotic cavity. Identification of biomarkers that indicate fetal lung maturation can inform a decision regarding the advisability of premature delivery.

In pregnancies complicated with intra-amniotic inflammation, inflammatory cells present in the amniotic fluid are for the most part of fetal and not of maternal origin. In this situation, the amniotic fluid contains neutrophils of fetal origin. The concentrations of inflammatory mediators in amniotic fluid that find there way into the vagina can predict the likelihood of impending preterm delivery and adverse neonatal outcome better than maternal blood in pregnancies complicated by intra-amniotic inflammation. As a result, amniotic fluid contains a large number of proteins that can act as diagnostic biomarkers of intra-amniotic and fetal inflammation.

Proteomic analysis of the protein composition of amniotic fluid thus provides a basis for diagnosing may aspects of fetal health and integrity and health of the amnion. Presently available technology can be used without extensive manipulation to practice the present invention. In one embodiment, the invention uses multi-track immunoassays, e.g., multi-track ELISAs to yield information that correlates with the existence of one or more abnormal clinical conditions. In other embodiments, SELDI analysis of vaginal fluid is used to yield this information. The present invention provides as well a means of assessing the extent and/or duration of some abnormal clinical conditions, by assaying the level of oxidized or carbonylated proteins in the vaginal fluid, and this aspect. Here again, either a conventional immunoassay format or SELDI analysis can be employed. A protein profile identified in accordance with the invention thus reliably indicates the presence or absence of inflammation, and its duration and/or intensity, which is of major importance because, as noted above, intra-amniotic inflammation is a risk factor for preterm delivery, short-term complications of prematurity, and long-term sequelae such as cerebral palsy and chronic lung disease.

The protein profile can form the basis of a recommendation for treatment. For example, the generated protein profiles can be combined with molecular microbiological techniques to identify microorganisms that are responsible for detected inflammation, thereby to inform selection of an antimicrobial therapy. In particular, samples of amniotic fluid from a patient determined to have intra-amniotic fluid in the vagina can be cultured in order to identify pathogenic microorganisms responsible for the inflammation. The cultures then can be tested to determine which antibiotics are effective against the identified microorganisms. While the profile in some situations may implicate antibiotic, tocolytic, anti-inflammatory, or antioxidant treatment, in other situations the profile may inform a decision to induce labor or perform a cesarean section. For example, it has been demonstrated in a mouse model that maternal ingestion of an anti-oxidant reduces the risk of preterm delivery secondary to infection. The presence of carbonylated proteins would indicate the need for combined therapy. Once an abnormal clinical status has been confirmed, continued analysis of vaginal samples can be performed to assess both the progression of the condition and the efficacy of any treatment.

In addition to monitoring the abnormal clinical status, tests for biomarkers of fetal health and maturity would also be undertaken once an abnormal intra-amniotic clinical status was confirmed. The results of these tests help to inform a decision whether pregnancy should be prolonged based on fetus viability.

Proteomic analysis of amniotic fluid, in accordance with the invention, provides a rapid, simple and reliable means of identifying the patient in premature labor with intra-amniotic inflammation, who are at risk for impending preterm delivery. Thus identified, this cohort of patients may be selected to test specific interventions to eradicate infection and/or to modulate the inflammatory response associated with adverse outcome.

The detection of biomarkers for the assessment of fetal health and maturity and the integrity and health of the amnion in a subject entails contacting a sample of vaginal fluid from a patient, with a substrate having an adsorbent thereon under conditions that allow binding between the biomarker and the adsorbent, and then detecting the biomarker bound to the adsorbent. In the case where the biomarker is an oxidized or carbonylated protein, the protein must first be derivatized with a group such as dinitrophenol that can be measured to qualify and/or quantify the presence of carbonyl groups on the protein.

Immunoassays can be used to detect any of the biomarkers according to the invention, alone or in combination with other of the biomarkers. A vaginal sample suspected of containing the biomarker(s) is mixed with an antibody to the biomarker(s) and monitored for biomarker-antibody binding. The biomarker is labeled with a radioactive or enzyme label. In a preferred embodiment, antibody for the biomarker is immobilized on a solid matrix such that it is accessible to biomarker contacting a surface of the matrix. The sample then is brought into contact with the surface of the matrix, and the surface is monitored for biomarker-antibody binding.

For example, a plurality of biomarkers can be identified by a multi-track enzyme-linked immunosorbent assay (ELISA), in which each track contains an antibody for one of the biomarkers bound to a solid phase. An enzyme-biomarker conjugate is used to detect and/or quantify the biomarker present in a sample. Alternatively, a Western blot assay can be used in which solubilized and separated biomarker(s) are bound to nitrocellulose paper. The biomarker then is detected by an enzyme or label-conjugated anti-immunoglobulin (Ig), such as horseradish peroxidase-Ig conjugate by incubating the filter paper in the presence of a precipitable or detectable substrate. Western blot assays have the advantage of not requiring purity greater than 50% for the desired biomarker(s). Descriptions of ELISA and western blot techniques are found in Chapters 10 and 11 of Ausubel, et al. (eds.), CURRENT PROTOCOLS IN MOLECULAR BIOLOGY, John Wiley and Sons (1988).

Alternatively, the biomarkers can be qualified and/or quantified using gas phase ion spectrometry, preferably by mass spectrometry and, in particular, by Surface Enhanced Laser Desorption and Ionization (SELDI). SELDI offers certain advantages over immunoassay methods. For example, SELDI can be done with a smaller sample of fluid, particularly where multiple biomarkers are being assessed. In order to profile multiple biomarkers in a sample with an ELISA or other immunoassay method requires multiple sample runs with different immunoassays, each of which requires an antibody that is specific to one of the biomarkers. SELDI, on the other hand, can be used to generate a profile for multiple biomarkers with a single small sample. The detection of the biomarkers can be enhanced by using certain selectivity conditions, e.g., adsorbents or washing solutions. A substrate comprising a suitable adsorbent can be in the form of a probe, which can be inserted into a gas phase ion spectrometer, preferably a mass spectrometer, or a substrate comprising the adsorbent can be mounted onto another substrate to form a probe that is inserted into the spectrometer.

The substrate with the adsorbent is contacted with the sample for a period of time sufficient to allow the biomarker to bind to the adsorbent. After the incubation period, the substrate is washed to remove unbound material. Any suitable washing solutions can be used. Preferably aqueous solutions are used. The washing solution can be determined by those of skill in the art.

An energy absorbing molecule then is applied to the substrate with the bound biomarkers. An energy absorbing molecule is a molecule that absorbs energy from an energy source in a gas phase ion spectrometer, thereby assisting in desorption of biomarkers from the substrate. Exemplary energy absorbing molecules include cinnamic acid derivatives, sinapinic acid and dihydroxybenzoic acid. Preferably sinapinic acid is used.

The biomarkers bound to the substrates are detected in a gas phase ion spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined.

Data generated by desorption and detection of markers can be analyzed with the use of a programmable digital computer. The computer program analyzes the data to indicate the number of markers detected, and optionally the strength of the signal and the determined molecular mass for each biomarker detected. Data analysis can include steps of determining signal strength of a biomarker and removing data deviating from a predetermined statistical distribution. For example, the observed peaks can be normalized, by calculating the height of each peak relative to some reference. The reference can be background noise generated by the instrument and chemicals such as the energy absorbing molecule which is set as zero in the scale.

The computer can transform the resulting data into various formats for display. The standard spectrum can be displayed, but in one useful format only the peak height and mass information are retained from the spectrum view, yielding a cleaner image and enabling biomarkers with nearly identical molecular weights to be more easily seen. In another useful format, two or more spectra are compared, conveniently highlighting unique biomarkers and biomarkers that are up- or down-regulated between samples. Using any of these formats, it can be readily determined whether a particular biomarker is present in a sample.

Software used to analyze the data can include code that applies an algorithm to the analysis of the signal to determine whether the signal represents a peak in a signal that corresponds to a biomarker according to the present invention. The software also can subject the data regarding observed biomarker peaks to classification tree or ANN analysis, to determine whether a biomarker peak or combination of biomarker peaks is present that indicates a diagnosis of intra-amniotic inflammation.

In another aspect, the present invention provides kits for aiding in the assessment of fetal health and maturity and the integrity and health of the amnion, which kits are used to detect biomarkers according to the invention. The kits screen for the presence of biomarkers and combinations of biomarkers that are differentially present in samples from normal subjects and subjects with an abnormal clinical status. The kits also may screen for the presence of biomarkers that are indicative of fetal health or maturity.

A kit includes at least one substrate having an adsorbent thereon, in which the adsorbent is suitable for binding a biomarker according to the invention. The kit additionally may contain a washing solution, or instructions for making a washing solution, in which the combination of the adsorbent and the washing solution allows detection of the biomarker using gas phase ion spectrometry. The kit also may include instructions for suitable operational parameters in the form of a label or separate insert. For example, the instructions may inform a consumer how to collect the sample or how to wash the probe. The kit may further include a pure form of the biomarker for use as a standard. Kits used to measure the presence or amount of oxidized or carbonylated proteins additionally may comprise a separate container of dinitrophenylhydrazine.

In one embodiment, the kit includes at least two substrates having adsorbents thereon, for detecting at least two biomarkers indicative of status of the intra-amniotic environment or the health or maturity of a fetus. The two substrates may have the same or different adsorbents. For example, a kit may include a cation exchange biochip and hydrophobic adsorbent biochip.

In another embodiment, a kit of the invention may include a first substrate, comprising an adsorbent thereon, and a second substrate onto which the first substrate is positioned to form a probe, which can be inserted into a gas phase ion spectrometer. In another embodiment, an inventive kit may comprise a single substrate that can be inserted into the spectrometer.

A proteomic assessment for a patient presenting with symptoms of PROM and/or preterm labor would illuminate the following: the integrity of the fetal membranes, the health of the intra-amniotic environment, the health of the fetus, and the maturity of the fetus. In a preferred embodiment, a single biochip binds biomarkers indicative of rupture of fetal membranes, intra-amniotic inflammation, and fetal pulmonary maturity. In combination with clinical tests such as ultrasound and culturing of fluid samples to identify microbes and assess their sensitivity to various antibiotics, the prognosis for the patient and the fetus can be greatly improved.

The present invention is further described by reference to the following, illustrative examples.

### EXAMPLE 1

### Discovery of Biomarkers by Comparing Vaginal Samples from Patients with Normal and Abnormal Clinical Status

Biomarkers according to the present invention were identified by comparing mass spectra of samples derived from vaginal fluid from two groups of pregnant subjects, subjects with an abnormal clinical status and normal subjects. The subjects were diagnosed according to standard clinical criteria. The two different pools of samples enable a differential analysis. Alternatively, surrogate samples were produced in vitro. For example, to confirm intra-amniotic bleeding the amniotic fluid profile in patients with an amniotic fluid red blood cell count over 5000 cells/mm³ was intersected with a diluted red blood cell lysate obtained from umbilical cord blood (fetal origin).

The two sample pools were used in a wide range of dilutions to test various biochip surfaces, produced by Ciphergen Biosystems, Fremont, Calif., for optimal discriminatory performance, including reverse phase H4, a hydrophobic surface with C-16 long chain aliphatic residues; SAX 2, a strong anion exchanger; WCX2, a quaternary ammonium, weak cation exchanger; IMAC, carboxylate residues; metal affinity). For H4 biochip surfaces, optimization involved additional hydrophobic washes of acetonitrile gradients (10% to 75%). A procedure in which 2 μl of vaginal fluid was diluted 10-fold in phosphate buffer saline (PBS), placed on a spot of a 24-spot H4 or WCX array, and incubated in a humidified box to avoid desiccation was found to be optimal for detection of peaks for beta-2-microglobulin and cystatin C, respectively. However, other biochips can be used, as long as they have the binding characteristics suitable for binding for the marker of interest. For beta-2-microglobulin and cystatin C, the preferred affinity surface comprises a hydrophobic adsorbent or a cation exchange adsorbent, respectively. For calgranulins and defensins, the preferred affinity surface is a hydrophobic adsorbent such as the Ciphergen H4 probe or H50 probe. For HCG the preferred affinity surface is an anion exchange chip, such as SAX.

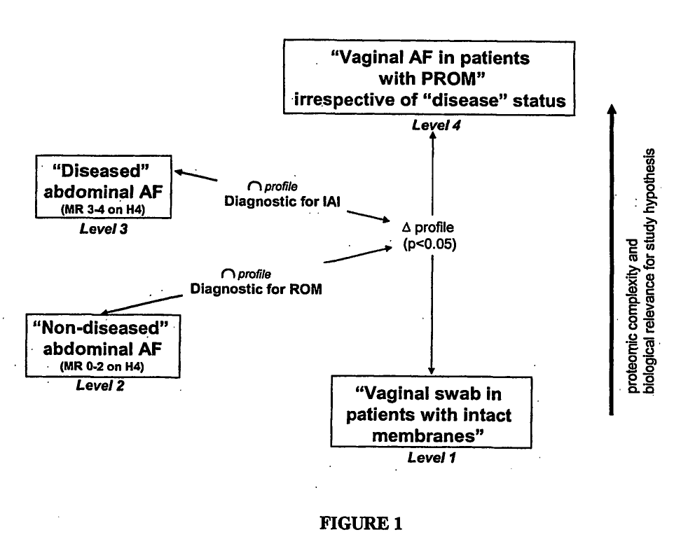

FIG. 1 shows the generalized model used to identify a biomarker profile in amniotic fluid obtained non-invasively from the vagina. In this instance, Level 0 is included in Level 1. However, the differential profile between the vaginal secretions in women with intact membranes (level 1) and PBS (level 0) will provide the normal vaginal proteome.

To define level 1, the vaginal proteome in pregnant patients with intact membranes, vaginal secretions were collected from patients with intact membranes using a cotton swab. The cotton swab was inserted into the posterior fornix of the vagina and then immersed in a centrifuge tube containing 0.5 ml of sterile phosphate buffer saline (PBS). After 1 minute, the solution in the vial was centrifuged and the supernatant analyzed.

After rupture of the membranes, amniotic fluid that leaked into the vagina could be collected and then diluted 1:10 by placing it into solution. The concentration of beta-2 microglobulin, cystatin C and alpha-fetoprotein was determined using specific ELISA assays. Beta-2 microglobulin, cystatin C and alpha-fetoprotein were selected based on earlier determination that they are present in amniotic fluid at high concentrations.

SELDI analysis of vaginal fluid on an H4 surface showed that beta-2 microglobulin had a conspicuous peak present in all fluids analyzed, and this was initially used as a peak reference (referred to as R peak). The R peak was matched to beta-2-microglobulin using an in-gel tryptic digest of the excised gel band and LC/MS/MS analysis.

SELDI analysis of vaginal fluid on a WCX surface showed that cystatin C also had a conspicuous peak in all samples. Cystatin C is an inhibitor of cysteine proteinase. It has a molecular weight of 13,347 Da and an isoelectric point of 8.75.

Neither the beta-2-microglobulin peak nor the cystatin C peak was seen in women who had intact membranes or who were not pregnant. Thus, the presence of these proteins on a vaginal swab is evidence of ruptured membranes, while their absence is evidence of or intact membranes.

The biochips were analyzed under protocols previously described in detail in 60/426,096, and the M and MR scores are calculated. In order to calculate an M score, conspicuous peaks are selected manually and the spectra from each patient are verified for accuracy of peak identification. The m/z value, normalized intensity, and signal-to-noise ratio (S/N) for the selected peaks are extracted. In a stepwise strategy based on Boolean logic, a diagnostic proteomic profile is established. Only the peaks obtained from the visual inspection process were subjected to stepwise analysis.

The criteria for the stepwise analysis included the following: (1) peaks present only in “diseased” patients were potential biomarker candidates, rather than the disappearance or decrease of peaks normally present in “non-diseased” patients; (2) only peaks of the profile detected on at least two different laser intensities or matrix protocols were potential biomarker candidates; (3) only peaks in the profile that were significantly different were potential biomarker candidates (as measured by the logarithm of normalized intensity, at least at a level of p<0.0001 between the “diseased” and “non-diseased” groups); (4) only parent peaks were potential biomarker candidates (singly ionized, least oxidized); (5) peaks that occurred in areas where the “noise” in “non-diseased” individuals was significantly elevated were eliminated as candidates; and (6) the number of peaks in the final diagnostic profile was kept to a minimum.

After applying the first four criteria, thirteen candidate biomarker peaks with potential discriminatory value emerged. To objectively score the peaks as present or absent, the evaluation of the S/N ratio was undertaken. The cut-off used for selection was the mean +2 standard deviations of the S/N ratio for each corresponding mass in the “non-diseased” group. Boolean indicators were then assigned: a value of 0 was used if a peak was absent or below the cut-off and a value of 1 was assigned for peaks above the cut-off. The sum of Boolean indicators was computed for each patient and is referred as the M score (Mass score).

The sum of 0 or 1 values for each peak results in an MR score (mass restricted score). When two peaks are profiled, such as beta-2-microglobulin and cystatin C, the MR score ranges from 0 to 2 depending upon the presence or absence of the two protein biomarkers. A categorical value of 1 is assigned if a particular peak is present and 0 if absent. Presence or absence of peaks is interpreted subjectively or may be calculated objectively relative to the readings from the PBS spots from the signal/noise ratio at the expected mass values. The final MR score is the summation of all the indicators (0=neither peak is present, 2=both peaks present). As validated by us in prior studies the MR score per se provides qualitative information regarding the presence or absence of an abnormal intra-amniotic status. A score of 2 indicates the presence of intra-amniotic abnormality, while a score of 0 signals a normal intra-amniotic environment. Thus, a quick visual inspection for absence or presence of defined peaks composing the MR score allows a person to calculate an MR score and establish a diagnosis.

Other biomarkers can be identified in accordance with the teachings herein, and these other biomarkers may best be profiled using other biochip surfaces. Biomarkers other than beta-2-microglobulin and cystatin C may be more readily detected with other biochip formats, which are easily determined by testing a wide range of dilutions on various biochip surfaces. Calgranulins and defensins, as described above, are best identified on an H4 biochip or similar, hydrophobic adsorbent-format biochip.

### EXAMPLE 2

### Discovery of Biomarkers by Comparing Vaginal Samples from Patients with Normal and Abnormal Clinical Status with Contemporaneously-Obtained Samples of Amniotic Fluid Via Amniocentesis

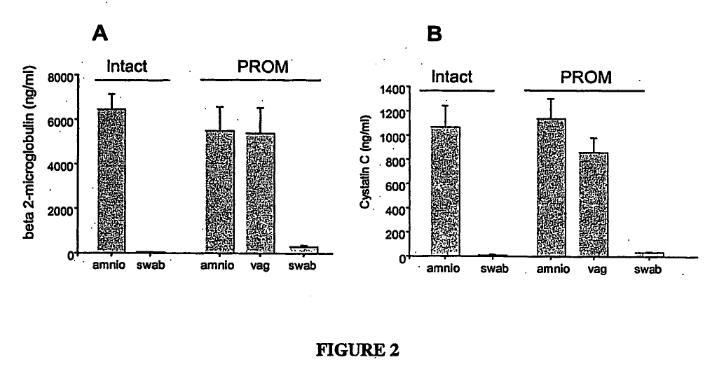

Samples obtained by a swab of the vagina of patients with intact or ruptured membranes and samples of amniotic fluid obtained contemporaneously by amniocentesis from the same patient were compared using specific ELISA assays. The results are shown in FIG. 2. When a swab saturated with amniotic fluid is immersed in 0.5 ml PBS the concentration in swab fluid of beta-2 microglobulin is 10% of the concentration in the pure fluid. This demonstrates that in women with ruptured membranes the vaginal environment contains biomarkers of amniotic cavity origin, such as beta-2-microglobulin and cystatin C, in a similar concentration to that found in the amniotic fluid obtained by amniocentesis. These biomarkers are absent in the vagina of women with intact membranes. Results with the SELDI platform showed the same close relationships noted in the ELISA studies, and confirmed that the analysis of vaginal pool on SELDI platform can easily identify a patient with ruptured membranes from a patient with intact membranes.

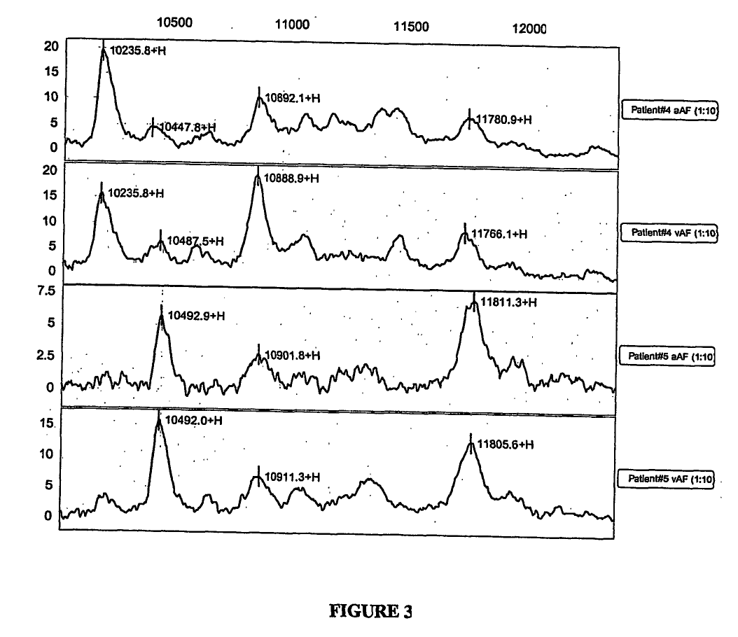

When intra-amniotic inflammation is present in women with PROM, the characteristic profile is seen in the vaginal pool. FIG. 3 illustrates four protein profiles from two women with PPROM and intra-amniotic inflammation. The findings confirm the potential of noninvasively obtaining an inflammatory profile from women with PROM using amniotic fluid present in the vagina. Subtraction of the protein profile from a swab of vaginal fluid from women with intact membranes reveals the inflammation biomarker peaks.

### EXAMPLE 3

### Serial Monitoring of Intra-Amniotic Inflammation

Serial samples of vaginal fluid from pregnant women were obtained and profiled by SELDI. This serial sampling successfully identified the appearance of inflammatory biomarkers indicative of incipient intra-amniotic inflammation before there was any clinical evidence. A normal profile obtained from the patient's vagina before rupture was subtracted to allow identification of the amniotic fluid peaks characteristics of inflammation, such as beta-2-microglobulin, cystatin C, alpha-fetoprotein. Even though the fluid became sparse (anhydramnios) as time passed, and the vaginal fluid became more viscous, profiling of samples by SELDI was possible. In this case, the sample was obtained by washing the syringe used for sampling with PBS.

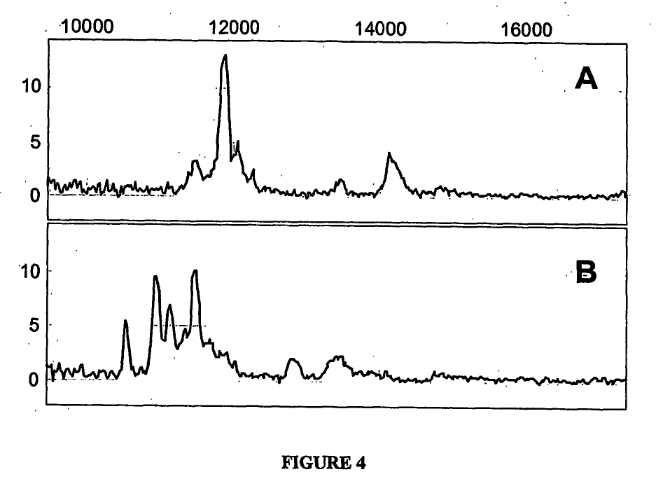

FIG. 4 shows a protein profile obtained from vaginal fluid of a patient monitored serially. On the day of admission, her normal profile was dominated by a high beta 2-microglobulin peak (upper tracing). Three days after rupture, her vaginal protein profile had changed dramatically showing dominant inflammatory peaks of S100 proteins and a suppressed beta-2-microglobulin peak. This patient was induced 6 days post rupture for clinically evident chorioamnionitis despite treatment with multiple antibiotics (ampicillin, gentamicin, metronidazole, and azithromycin). A pathological change consistent with fetal inflammation thus was identified with SELDI three days prior to clinical signs of chorioamnionitis. Moreover, it should noted that in this patient, an attempted amniocentesis on admission was unsuccessful with no fluid withdrawn because of anhydramnios. This occurs in as many as 20% of women with preterm premature rupture of membranes. Thus, profiling of a non-invasive vaginal sample according to the invention was successful where amniocentesis was not.

### EXAMPLE 4

### Detection of Oxidized or Carbonylated Proteins in Samples of Vaginal Fluid

An experiment was undertaken to assess the correlation between the amount of oxidants, time of exposure and extent of protein oxidation as estimated by protein carbonylation in amniotic fluid. In this experiment amniotic fluid was exposed to peroxyl free radicals generated in vitro by the spontaneous decomposition of 2,2′ azobis-2-methylpropionamidine dihydrochloride (ABPA 30, 60, 90, 120, 150 mM). Protein carbonylation and protein fragmentation were assessed as indicators of free radical damage. Total carbonyl content was measured by derivatization with dinitrophenylhydrazine (DNPH) followed by Western blotting for DNPH-derivatized proteins using anti-dinitrophenyl DNP antibodies before and after 1, 36, 24 h of exposure to ABPA. Protein fragmentation was quantified by the decrease in the intensity of the serum albumin band on Coomasie or silver stained gels loaded with similar amounts of protein. A dose and time-dependent increase in protein carbonylation in amniotic fluid in response to ABPA was observed. Protein carbonylation was associated with substantial protein degradation, and this was substantially higher in amniotic fluid than in fetal plasma.

Protein oxidation or carbonylation in amniotic fluid is present in intra-amniotic inflammation. The median amount of DNP is an indicator of the extent of protein carbonylation. This was significantly greater in patient groups with intra-amniotic inflammation than in patient groups without intra-amniotic inflammation (median intra-amniotic inflammation: 62.5 [5-95% percentiles: 15-112] fmols DNP/μg protein versus preterm no inflammation 20.5 [8-42] fmols DNP/μg protein versus term C/S: 27 [12-44] fmols DNP/μg protein). Thus, the preterm fetus of a mother with intra-amniotic inflammation is exposed to a highly oxidative environment. Furthermore, when intra-amniotic inflammation was estimated by MR score, protein oxidation was significantly higher in samples of amniotic fluid that had MR scores greater than 2. These data show that protein oxidation is a valuable source of information about the extent and gravity of the inflammatory insult that may relate better to fetal outcome than the presence of inflammation per se. A DNP antibody capture assay or SELDI analysis of biomarkers of oxidized or carbonylated proteins thus may be used to complement the identification of biomarkers that indicate the presence of inflammation.

What is claimed is:

1. A method for assessment of the intra-amniotic environment, comprising (A) obtaining a vaginal sample from a subject, and (B) subjecting the sample to analysis, to determine presence or absence in the sample of a plurality of biomarkers that is indicative of status of the intra-amniotic environment, such that results from the assessment of the vaginal sample informs a diagnostic or prognostic determination in relation to the subject.

2. The method as claimed in claim 1, wherein (A) and (B) are repeated at least at a second time.

3. The method as claimed in claim 1, wherein the biomarkers are indicative of rupture of the fetal membrane.

4. The method as claimed in claim 1, wherein the biomarkers are indicative of intra-amniotic infection.

5. The method as claimed in claim 1, wherein the biomarkers are indicative of intra-amniotic inflammation.

6. The method as claimed in claim 1, wherein the biomarkers are indicative of fetal lung maturation.

7. The method as claimed in claim 1, wherein the biomarkers are selected from the group consisting of alpha-fetoprotein, fetal fibronectin, insulin-like growth factor binding protein-1, prolactin and human placental lactogen, and fragments thereof.

8. The method as claimed in claim 1, wherein the biomarkers are selected from the group consisting of beta-2-microglobulin and cystatin-C, and fragments thereof.

9. The method as claimed in claim 1, wherein the plurality of biomarkers is subjected to pattern recognition analysis.

10. The method as claimed in claim 1, wherein the method is an ELISA.

11. The method as claimed in claim 1, wherein the method comprises mass spectrometric analysis effected via SELDI.

12. The method as claimed in claim 11, wherein the method comprises applying the vaginal sample to a biochip comprising at least one absorbent selected from the group consisting of a hydrophobic adsorbent and a cation exchange absorbent.

13. The method as claimed in claim 11, the mass spectrometric analysis comprises subjecting mass-spectrometry peak data obtained for the vaginal sample to software analysis comprised of an algorithm for analyzing data extracted from a spectrum.

14. The method as claimed in claim 13, wherein the algorithm implements a pattern-recognition analysis that is keyed to data relating to at least one of the biomarkers.

15. The method as claimed in claim 1, wherein a first vaginal sample is collected early during a pregnancy and contributes to a baseline against which subsequent vaginal samples are compared.

16. The method as claimed in claim 15, wherein the determination includes a recommendation for treatment.

17. The method as claimed in claim 16, further comprising monitoring the treatment by assaying at least one vaginal sample during treatment, to determine the presence or absence in the vaginal sample of biomarkers that are indicative of status of the intra-amniotic environment.

18. The method as claimed in claim 16, wherein the determination includes a recommendation of treatment that comprises antibiotic treatment, tocolytic treatment, anti-inflammatory treatment, or antioxidant treatment.

19. The method as claimed in claim 16, wherein the determination includes a recommendation of treatment that comprises inducing labor.

20. The method as claimed in claim 16, wherein the determination includes a recommendation of treatment that comprises a cesarean section.

21. A method for assessment of the intra-amniotic environment, comprising (A) obtaining a vaginal sample from a subject, (B) subjecting the sample to analysis, to determine the presence or absence in the sample of one or more oxidized or carbonylated peptides that are indicative of status of the intra-amniotic environment, such that results from the assessment of the vaginal sample informs a diagnostic or prognostic determination in relation to the subject.

22. The method as claimed in claim 21, wherein the vaginal sample is treated with dinitrophenol which is incorporated into the oxidized or carbonylated peptide.

23. The method as claimed in claim 21, wherein the method is an ELISA.

24. The method as claimed in claim 21, wherein the method comprises mass spectrometric analysis effected via SELDI.

25. The method as claimed in claim 21, wherein the method comprises applying the vaginal sample to a biochip comprising at least one absorbent selected from the group consisting of a hydrophobic adsorbent and a cation exchange absorbent.

26. The method as claimed in claim 24, wherein the mass spectrometric analysis comprises subjecting mass-spectrometry peak data obtained for the vaginal sample to software analysis comprised of an algorithm for analyzing data extracted from a spectrum.

27. The method as claimed in claim 26, wherein the algorithm implements a pattern-recognition analysis that is keyed to data relating to a plurality of oxidized or carbonylated peptides.

28. The method as claimed in claim 21, wherein a plurality of oxidized or carbonylated peptides is subjected to pattern recognition analysis.

29. The method as claimed in claim 21, wherein a first vaginal sample is collected early during a pregnancy and contributes to a baseline against which subsequent vaginal samples are compared.

30. The method as claimed in claim 21, wherein the determination includes a recommendation for treatment.

31. The method as claimed in claim 30, further comprising monitoring the treatment by assaying at least one vaginal sample during treatment, to determine the presence or absence in the vaginal sample of the one or more oxidized or carbonylated peptides.

32. The method as claimed in claim 30, wherein the determination includes a recommendation of treatment that comprises antibiotic treatment, tocolytic treatment, anti-inflammatory treatment, or antioxidant treatment.

33. The method as claimed in claim 30, wherein the determination includes a recommendation of treatment that comprises inducing labor.

34. The method as claimed in claim 30, wherein the determination includes a recommendation of treatment that comprises a cesarean section.

35. The method as claimed in claim 22, wherein the treated vaginal sample is applied to a biochip comprising an anti-dinitrophenol antibody and subjected to mass spectrometric analysis that is keyed to a shift in molecular weight, relative to a sample not treated with dinitrophenol, that corresponds to the incorporated dinitrophenol group.

36. The method as claimed in claim 22, wherein the treated vaginal sample is applied to a biochip comprising an anti-dinitrophenol antibody and subjected to mass spectrometric analysis is keyed to a shift or approximately 16 Da, relative to a sample not treated with dinitrophenol, that corresponds to the molecular mass of oxygen.

37. The method as claimed in claim 21, wherein total carbonyl content of the oxidized or carbonylated peptides is measured by derivatizing the peptides with dintrophenylhydrazine.

38. A method for qualifying status of the intra-amniotic environment in a subject over time, comprising (i) providing spectra generated by mass spectrometric analysis of at least two vaginal samples taken from the subject, and (ii) extracting data from the spectra and subjecting the data to pattern-recognition analysis that is keyed to at least two peaks in the spectra.

39. A kit for detecting, from a sample of vaginal fluid, the presence of at least two biomarkers indicative of status of the intra-amniotic environment, comprising (a) a substrate adapted for inserting into a mass spectrophotometer for analysis, and (b) instructions for applying a sample of vaginal fluid to the substrate and subjecting the substrate to mass spectrometric analysis.

40. The kit as claimed in claim 39, wherein the substrate is a biochip.

41. The kit as claimed in claim 40, wherein the biochip comprises at least one absorbent selected from a hydrophobic adsorbent and a cation exchange adsorbent.

42. The kit as claimed in claim 40, wherein the biochip comprises an anti-dinitrophenol absorbent.

43. The kit as claimed in claim 39, additionally comprising, in a separate container, a quantity of the biomarker in pure form to be used as a standard.

44. The kit as claimed in claim 43, a washing solution for removing unbound material from the substrate.

45. A kit for detecting, from a sample of vaginal fluid, the presence of at least one oxidized or carbonylated peptide indicative of status of the intra-amniotic environment, comprising (a) a substrate that binds the peptide, and (b) instructions for applying a sample of vaginal fluid to the substrate and subjecting the substrate to analysis.

46. A kit as claimed in claim 45, comprising an ELISA substrate.

47. The kit as claimed in claim 45, comprising a substrate adapted for insertion into a mass spectrophotometer for analysis.

48. The kit as claimed in claim 45, additionally comprising, in separate container, a quantity of the oxidized or carbonylated peptide in pure form to be used as a standard.

49. The kit as claimed in claim 48, a washing solution for removing unbound material from the substrate.

50. A method for identifying biomarkers that are present in vaginal fluid and are indicative of status of the intra-amniotic environment, comprising:
(a) profiling a sample of vaginal fluid by mass spectrophotometric analysis,
(b) profiling a sample of amniotic fluid by mass spectrophotometric analysis, and
(c) comparing the profiles obtained in (a) and (b) to identify biomarkers in vaginal fluid that also are found in amniotic fluid.

51. The method as claimed in claim 50, additionally comprising correlating the presence or absence of the biomarkers in the vaginal fluid that are also found in the amniotic fluid to a clinical status.

52. The method as claimed in claim 51, wherein the clinical status is rupture of the fetal membrane.

53. The method as claimed in claim 51, wherein the clinical status is intra-amniotic infection.

54. The method as claimed in claim 51, wherein the clinical status is intra-amniotic inflammation.

55. A method for identifying biomarkers that are present in vaginal fluid and are indicative of status of the intra-amniotic environment, comprising:
(a) profiling a first sample of vaginal fluid from a subject having a normal pregnancy by mass spectrophotometric analysis,
(b) profiling a second sample of vaginal fluid from a subject having a pregnancy characterized by an abnormal clinical status by mass spectrophotometric analysis, and
(c) correlating the presence or absence of the biomarkers in the vaginal fluid to clinical status of the pregnancy.
